(19) United States
(12) Patent Application Publication
(10) Pub. No.: US 2022/0105257 A1
(43) Pub. Date: Apr. 7, 2022

(54) Rectal Irrigation Apparatus

(71) Applicant: Guy Merz, Glendale, AZ (US)

(72) Inventor: Guy Merz, Glendale, AZ (US)

(21) Appl. No.: 17/063,422

(22) Filed: Oct. 5, 2020

(51) Int. Cl. A61M 3/02

(52) U.S. Cl. CPC A61M 3/025; A61M 3/0283; A61M 2202/068; A61M 2206/16; A61M 2205/3389; A61M 2210/1067

(57) ABSTRACT

A rectal irrigation apparatus for irrigating a patient's rectum with irrigation fluid (also called lavage) and evacuating feces therefrom. The apparatus comprises several components that work together for integrated operation. The spray nozzle is designed to swirl the spray irrigation fluid in a vortex motion to break-up or loosen feces in the rectum. For example, the spray nozzle may contain a spinning auger to create the vortex motion. The fecal waste is suctioned out into a waste container. The apparatus further comprises a control module that integrates the operation of the various components, such as the vortex generator, the water pump, suction pump, or toilet mechanism. This apparatus could be particularly useful in the treatment of fecal impaction.

### TECHNICAL FIELD

This invention relates to rectal irrigators that use liquid pumped from a reservoir.

### BACKGROUND

Many people, particularly those who are elderly or handicapped, have substantial difficulty with bowel movements or have lost the ability to voluntarily evacuate their bowels. This can result in a condition called “fecal impaction” in which the feces have become hardened and cannot be evacuated by a bowel movement. This is a condition that requires medical attention. Conventional treatment for fecal impaction include enemas or manual removal of feces. However, conventional enema treatments are often insufficient for serious cases of fecal impaction. Moreover, conventional enemas do not provide a means for collecting the fecal waste.

For more advanced treatment, colonic irrigation or lavage devices have been developed. One example is the PIE* (Pulsed Irrigation Evacuation) Device made by P.I.E. Medical LLC for irrigating and evacuating stool from entire colon. The PIE device uses pulsed irrigation of lavage fluid to loosen, break-up, and purge the fecal matter. A description of this apparatus is given in U.S. Pat. No. 6,106,506 (Abell). As described therein, the apparatus generates a pulsing action in the lavage water to break-up and dissolve impacted feces. However, there is a need for alternate designs for integrated, automatically-controlled apparatus for delivering a colonic lavage.

### SUMMARY

The present invention provides an apparatus for irrigating a patient's rectum with irrigation fluid (also called lavage) and evacuating feces therefrom. The apparatus comprises multiple components that work together for integrated operation. Spray Nozzle: The apparatus comprises a spray nozzle for spraying irrigation fluid into the patient's rectum. The spray nozzle comprises a nozzle body and a soft nozzle tip. The soft nozzle tip is designed for insertion into the patient's anus to deliver irrigation fluid into the rectum. The nozzle tip may comprise any suitable soft or rubber-like material such as silicone, fluoroelastomers, nitrile, or ethylene propylene. In some cases, the nozzle tip has a diameter of 4-20 mm. The spray nozzle could have a flow sensor to measure the flow rate through the spray nozzle. The spray nozzle could have a valve to control the flow rate of the spray.

Irrigation Fluid & Container: The apparatus further comprises an irrigation fluid container to hold and supply the irrigation fluid. The irrigation fluid container may be of any suitable volume (e.g. 1-5 liters). The irrigation fluid container could be transparent or semi-transparent to facilitate inspection of the fluid volume remaining. In some cases, the irrigation fluid container comprises a heating device. Any suitable heating device capable of warming the irrigation fluid to a temperature of 70°-130° F. could be used. In some cases, the irrigation fluid container comprises a temperature sensor to measure the temperature of the irrigation fluid.

Any suitable water-based fluid may be used as the irrigation fluid. The irrigation fluid could be plain water or other fluid conventionally used for body cavity irrigation, such as saline water. The irrigation fluid could contain additives such as detergents, emulsifiers, surfactants, or enzymes to help soften or break-up the feces.

Water Pump: The apparatus further comprises a water pump to pump the irrigation fluid out of the irrigation fluid container and into the spray nozzle. The water pump may be any conventional pump for pumping liquids. The water pump could be water-submersible. For example, the water pump may be positioned outside the irrigation fluid container or within the irrigation fluid container and submersed within the irrigation fluid. The water pump could have controls to adjust the irrigation flow rate. The water pump could be coupled to the control module.

Suction Pump: The apparatus further comprises a suction pump for suctioning fecal waste into the waste container. Any suitable type of gas suction pump (also known as vacuum pumps) could be used. The suction pump could have controls to adjust the level of vacuum pressure. The suction pump could be coupled to the control module.

Vortex Generator: The apparatus further comprises a vortex generator contained inside the nozzle body. There are a variety of mechanical designs that are possible to produce swirling motion in liquid fluid. For example, the vortex generator could be an auger, screw, swirl tip, swirl vanes, swirl chamber, spiral nozzle, spinning disks, etc. In some embodiments, the vortex generator is a spinning tool that is capable of inducing such vortex motion, such as an auger, propeller, shaft with rotating blades or vanes, screws, etc.

The vortex generator could operate by fluid pressure from the water pump, or by an electric motor, or combination thereof. The fluid turbulence from the vortex action causes breaking-up or loosening of feces. As used herein, the term ‘vortex action’ with respect to the spray does not mean just the direction of spray. The term also implicates the post-spray motion of the irrigation fluid. That is, simply spraying at a radial angle out the nozzle does not constitute ‘vortex motion’ as meant in this invention. There must also be swirling motion of the fluid after it exits out the nozzle. This vortex action is an improvement over pulsed water irrigations of the prior art which can be uncomfortable for the patient and less effective on fecal impactions. Vortex action has a much gentler agitation to the rectum. This vortex action alone or in combination with pulsed irrigation is more effective than pulsed irrigation alone.

Electric Motor: In some embodiments, the apparatus further comprises an electric motor for powering the vortex generator. The electric motor is connected to the vortex generator, for example, by a spinning shaft. Any suitable electric motor may be used in the apparatus. The electric motor could have controls to adjust the spin rate (i.e. rpm) or power. The electric motor may be coupled to the control module.

Control Module, Sensors, and Switches: The apparatus may have one or more control modules for operating the apparatus. The control module is coupled to the various components of the apparatus to control their operation, such as activation, deactivation, speed, intensity, duration, timing, flow rates, fluid pressure, or suction pressure, etc. The control module could be coupled to the other components of the apparatus by wired or wireless communications. Examples of wireless remote control include radio (such as WiFi or Bluetooth) or infrared light (IR). The control module may be a separately designed device, or may be an off-the-shelf device such as a smartphone, tablet computer, or laptop computer, or combination thereof.

The control module could be coupled to the various components of the apparatus, such as the vortex generator, spray nozzle, electric motor for the vortex generator, water pump for pumping irrigation fluid, suction pump, toilet flush actuator, heating device for the irrigation fluid, etc. For example, the control module could be coupled to the electric motor for the vortex generator to control the intensity of the vortex action, to the water pump to control the fluid irrigation flow rate, to the suction pump to control the amount of suction pressure being applied in the patient's rectum or the flow network.

The control module may have some preprogramed or programmable features. For example, certain routines (e.g. start-up or shutdown) could be pre-programmed. Examples of programmed or programmable settings that could be controlled by the control module include the procedure for irrigation (such as the total volume of irrigation fluid to be infused, flow rate of irrigation fluid, duration, etc.), procedure for suctioning waste (such as the pressure safety limits, vacuum suction pressure applied, duration, etc.), or procedure for waste flushing.

The apparatus may also have various sensors that detect a status of the apparatus or component thereof. The control module may be coupled to such sensors to receive status data and provide feedback control over the operation of the apparatus or components thereof. For example, the apparatus could have a flow rate sensor (e.g. in the spray nozzle, supply hose, or inflow port), temperature sensor in the irrigation fluid container to measure the temperature of the irrigation fluid, a pressure sensor somewhere in the suction flow network (e.g. an outflow port, waste container, or suction hose) to measure the amount of vacuum pressure being applied, fluid level or volume sensors to measure the amount of fluid in the irrigation fluid container or the waste container, etc. The control module may also be coupled to various valves, ports, or switches that may be part of the apparatus to control the opening, closing, or actuating of such components.

The control module can have control interface elements for the user. Examples of control interface elements include pushbuttons, number keys, keyboards, dials, knobs, touchscreens, switches, toggles, tuners, levers, adjusters, etc. The control module could also have one or more displays to show information. Various types of information could be displayed to the user, such as the progress of the irrigation procedure, fluid volume that has been pumped, present flow rate, time elapsed from the start of the procedure, or estimated time left, etc. Further, the display may guide the user about how to use the settings and functions of the apparatus. There may be one or more control units that constitute the control module for the apparatus. The control module may be a single unitary control unit or distributed over several control units.

Waste Container: The apparatus further comprises a waste container for receiving fecal waste. The waste container could be transparent or semi-transparent so that the filling of the waste container could be easily monitored. The waste container could have a fluid volume or fluid level sensor, such as a float switch, to detect the volume of waste filling the container. The waste container has connections for a discharge hose (incoming from the spray nozzle) and a suction hose (outgoing to the suction pump). The waste container could have a purge port at the bottom as an outlet for the fecal waste to flow out. This feature may be useful when the apparatus is used in conjunction with a flush toilet. That is, the purge port would allow the waste container to purge the waste directly into the toilet.

The waste container may also have other valves or ports. For example, the purge port could have a valve that opens to allow the drainage of the waste. The waste container could have an air inlet port to allow air entry and facilitate smooth flow of waste out of the purge port. The fluid volume/level sensor could be used to automate the purging process. The fluid volume/level sensor could be used to control the opening of the air inlet port or the opening of the purge port valve. For example, the fluid volume/level sensor could detect that the waste container is full, and cause both the air inlet port and the purge port valve to open. This control may be direct (e.g. by wires connected to the air inlet port mechanism and the purge port valve mechanism) or may be indirect, e.g. through the control module.

Support Frame & Other Components: The apparatus may further comprise a support frame to hold the components of the irrigation apparatus. The support frame may be designed to make the apparatus more mobile or portable, adaptable for bedside use, easy cleaning, easy disassembly, and maneuverability of components. The frame could be designed to hold the various machinery, tubing, power supply, or electrical wiring. For example, the frame may have a mounting arm upon which the spray nozzle could be mounted so that it could be maneuvered for insertion into the patient's anus. In another example, the frame could have tubing holders to organize the various tubes that may be used in the apparatus. In another example, the frame could have wiring harnesses to organize the various wiring that may be used in the apparatus. An example of a frame that could be used is described in U.S. Pat. No. 6,106,506 (Abell).

In some embodiments, the apparatus further comprises a colon catheter tube for positioning into the rectum or sigmoid colon (that is, the distal end of the colon catheter tube). In some embodiments, the colon catheter tube has a length in the range of 15-40 inches (38-102 cm). In some embodiments, the colon catheter tube has a width in the range of 12 French size (4 mm) to 40 French size (13.3 mm), measuring the outer diameter size. The colon catheter tube could have its own separate supply hose (e.g. accessory supply hose), or may be connected directly or indirectly to the main hose. For example, it could be supplied by a Y-connection off the main supply hose, or connected to an outlet port on the spray nozzle.

In some embodiments, the apparatus further comprises a platform for supporting the waste container over a toilet. The platform has a hole through which a purge port of the waste container passes therethrough. In some cases, the diameter of hole is in the range of 1-4 cm wide. The platform may have any suitable shape for fitting on top of a toilet bowl, such as square, rectangular, polygonal, round, or oval shape. The platform is sufficiently sized to fit on top of a toilet bowl. In some cases, platform has a length of at least 25 cm, a width of at least 25 cm, a diameter at least 25 cm, a widest dimension of at least 25 cm, or any combination thereof, depending on the shape of the platform. In some cases, the platform has a surface area of at least 500 cm². The platform is designed to be strong enough to support the waste container, but should also be sufficiently thin to allow easy handling. In some cases, the platform has a thickness of less than 1.5 cm. The platform could be made of any suitable material, including a variety of different types of plastics.

Various functional components of the apparatus could be separate or combined into a single unit. For example, the electric motor and the water pump could be combined into a single unit. In another example, the water pump and the suction pump could be combined into a single unit. In addition, various sensors and accessories could be combined into its counterpart components as a single functioning unit. The functions of the control module of the apparatus may be performed by a single unit or may be distributed among multiple (two or more) control units. For example, individual components of the apparatus (such as the pumps, electric motor, or waste container) could have its own control unit.

Rectal Irrigation Method: In another aspect, this invention is a method of performing rectal irrigation on a patient. This method may be performed with the rectal irrigation apparatus described herein, or any other suitable rectal irrigation apparatus. The method comprises inserting a soft nozzle tip of a spray nozzle into the rectum through the patient's anus. From the spray nozzle, irrigation fluid is sprayed into the rectum. The irrigation fluid may be sprayed out at any suitable flow rate. In some embodiments, the irrigation fluid flows out of the spray nozzle at a flow rate in the range of 400-1,500 ml/min.

The spraying is performed in a manner to induce vortex action in the sprayed irrigation fluid such that it has a swirling motion in the patient's rectum. This vortex action breaks up or loosens feces in the rectum. The method further comprises suctioning fecal waste out of the rectum into a waste container.

The waste container may be set over a toilet to allow easy disposal of the fecal waste. In some cases, the method comprises setting a platform on a toilet, wherein the platform has a hole through which a purge port of the waste container passes therethrough. The purge port is located at a bottom part of the waste container. The method further comprises setting the waste canister on the platform with the purge port passing through the hole.

In some cases, the irrigation fluid is heated or maintained at a temperature of 90°-120° F. to provide more comfort to the patient. In some cases, the generation of the vortex action in the sprayed irrigation fluid is started after a delay (e.g. at least 0.7 seconds) from starting the water pump. In some embodiments, the suctioning of fecal waste is started after a delay (e.g. at least 0.7 seconds) from starting the generation of the vortex action.

The method further comprises suctioning fecal waste out of the rectum and spray nozzle. The fecal waste is deposited into a waste container. Any suitable suction pressure may be used to suction the fecal waste out of the rectum. In some cases, the suction pressure is in the range of 5-100 kPa.

In some cases, the method further comprises stopping or reducing the flow rate of irrigation fluid, but continuing to maintain the vortex generation for at least a partial duration (e.g. at least one second) while the flow is stopped or slowed. In some cases, the vortex generation is maintained for at least a partial duration (e.g. at least one second) while the suctioning occurs.

In some cases, the method further comprises sensing the fluid level or volume in the waste canister. The method may further comprise, if the fluid level/volume exceeds a certain threshold, purging the fecal waste out of the waste canister into the toilet by gravity drainage. In some cases, the method may further comprise, if the fluid level/volume exceeds a certain threshold, opening a purge valve on the waste canister to allow discharging of fecal waste out of the waste canister into the toilet.

### BRIEF DESCRIPTION OF THE DRAWINGS

FIG. 1 shows a schematic layout of an example rectal irrigation apparatus of this invention.

FIGS. 2A-2C show an example of how the rectal irrigation apparatus could be operated. FIG. 2A shows the patient having a mass of hardened feces. FIG. 2B shows the results of the vortex irrigation spray. FIG. 2C shows the fragmented feces being evacuated out of the rectum.

FIGS. 3A-3C show another design for a rectal irrigation apparatus. FIG. 3A shows a detailed illustration of a design for a waste canister. FIG. 3B shows a perspective view of the waste canister and the platform on which it is mounted. FIG. 3C shows an example of how the fecal waste section of the apparatus could operate.

FIG. 4 shows a different example of a control module of the apparatus.

FIG. 5 shows a partial view of an alternate embodiment of the rectal irrigation apparatus that can provide a colonic enema via a colonic catheter in addition to rectal irrigation.

FIG. 6 shows a partial view of an alternate embodiment of the apparatus that has more useful features for an irrigation fluid container.

FIG. 7 shows another design for a waste container of this invention.

### DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

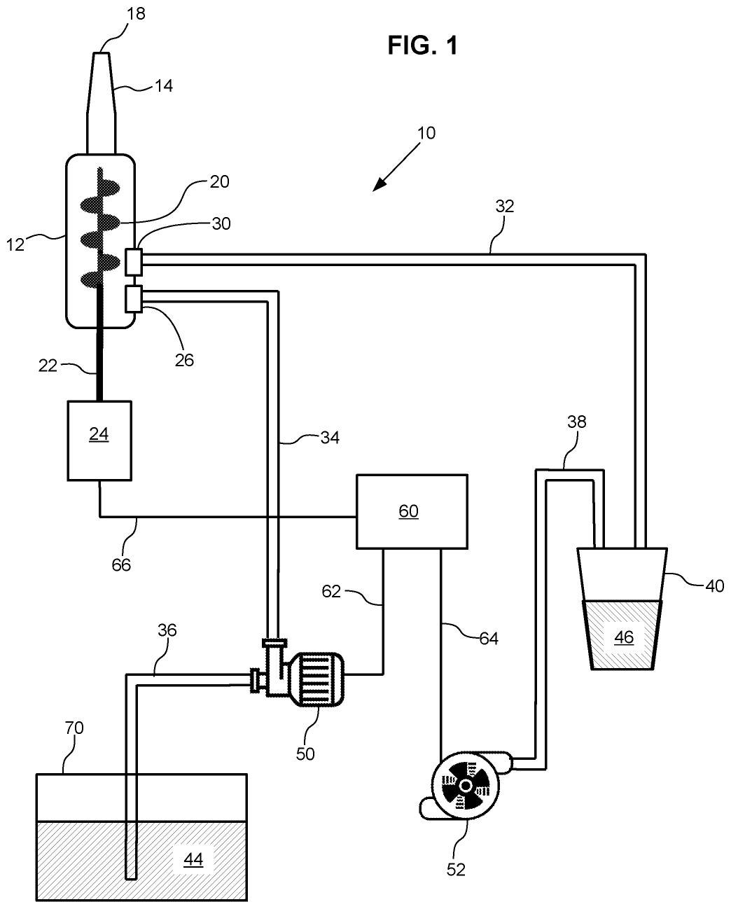

FIG. 1 shows a schematic layout of an example rectal irrigation apparatus 10 of this invention. At the distal end of the rectal irrigation apparatus 10 is a spray nozzle that comprises a nozzle body 12 and a soft insertion tip 14 for insertion into the patient's rectum via the anus. At the distal portion of the soft insertion tip 14 is a spray outlet 18. The nozzle body 12 is a sealed enclosure to contain an irrigation fluid (liquid). There is a continuous fluid passage from the nozzle body 12 through the insertion tip 14 out through spray outlet 18. Irrigation fluid enters the nozzle body 12 through an inflow port 26. Also, fecal waste exits the nozzle body 12 out through the outflow port 30.

Pumped irrigation fluid enters inside the nozzle body 12 of the spray nozzle via a supply hose 34 and connected to inflow port 26. The pumped irrigation fluid continues passing through the insertion tip 14 and is then sprayed out through spray outlet 18. Inside the nozzle body 12 is a helical augur 20, which is connected to an electric motor 24 via a turnshaft 22. Upon activation of the electric motor 24, the helical augur 20 is made to spin. This spinning of the helical augur 20 generates a vortex of the irrigation fluid inside the nozzle body 12. The vortexed agitation of irrigation fluid is maintained as it passes through the insertion tip 14 and then out of spray outlet 18.

The apparatus 10 further comprises a supply reservoir 70 containing the irrigation fluid 44 for supply to the spray nozzle. The apparatus 10 further comprises a water pump 50 that pumps the irrigation fluid 44 out of the supply reservoir 70 and pushes the irrigation fluid 44 into the nozzle body 12. The water pump 50 draws irrigation fluid 44 out of the supply reservoir 70 via a reservoir hose 36. The irrigation fluid 44 is driven by the water pump 50 into the nozzle body 12 via the supply hose 34. The supply hose 34 is connected to the inflow port 26. Thus, the pumped irrigation fluid 44 flows into supply hose 34, through inlet port 26, and into the nozzle body 12 of the spray nozzle.

The apparatus 10 further comprises a waste canister 40 for receiving the discharged fecal waste. The waste canister 40 is connected to a vacuum pump 52 via a suction hose 38. The waste canister 40 is also connected to the outflow port 30 of nozzle body 12 via a discharge hose 32. In operation, activation of the vacuum pump 52 creates suction inside the waste canister 40. This suction is transmitted through the suction hose 38, through waste canister 40, through discharge hose 32, through the outflow port 30, and into the nozzle body 12. This suction draws fecal waste out of the nozzle body 12, out through the discharge hose 32, and deposits the fecal waste into the waste canister 40.

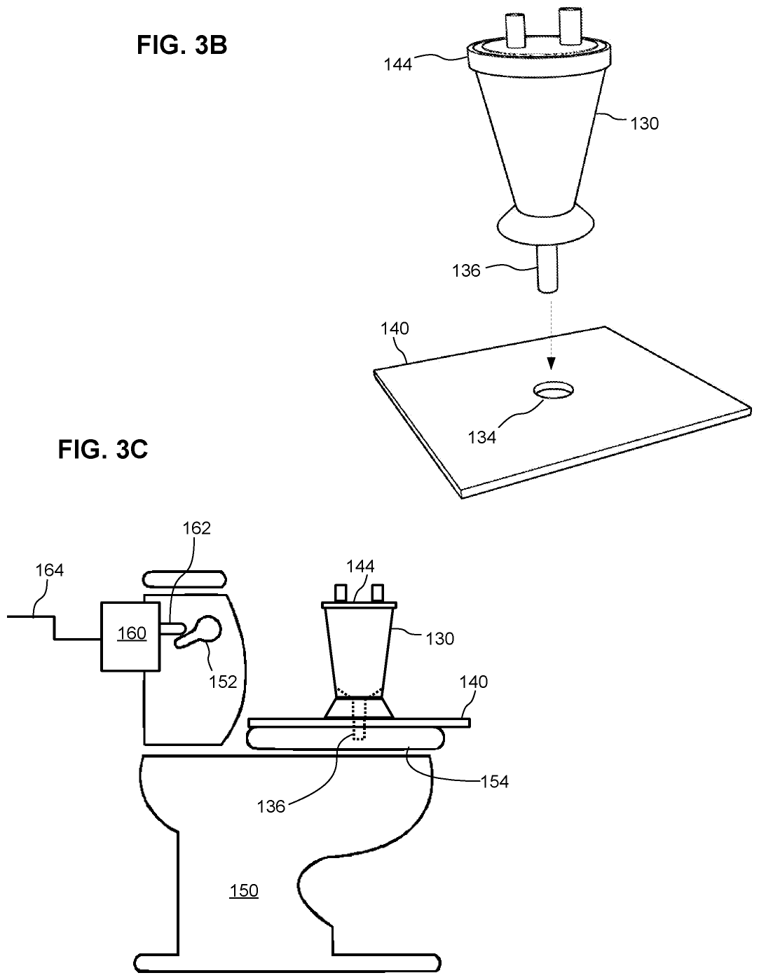

Operation of the water pump 50, the electric motor 24, and the vacuum pump 52 are controlled by a control module 60. The control module 60 has control lines (with wires) to the various components. Control line 66 connects the control module 60 to the electric motor 24. Control line 62 connects the control module 60 to the water pump 50. Control line 64 connects the control module 60 to the vacuum pump 52. The control module 60 has various user interface components for controlling the operation of the apparatus 10 (e.g. buttons, switches, display screens, touchscreens, etc.). A more detailed embodiment of a control module and its functions is shown in FIG. 4, as explained below.

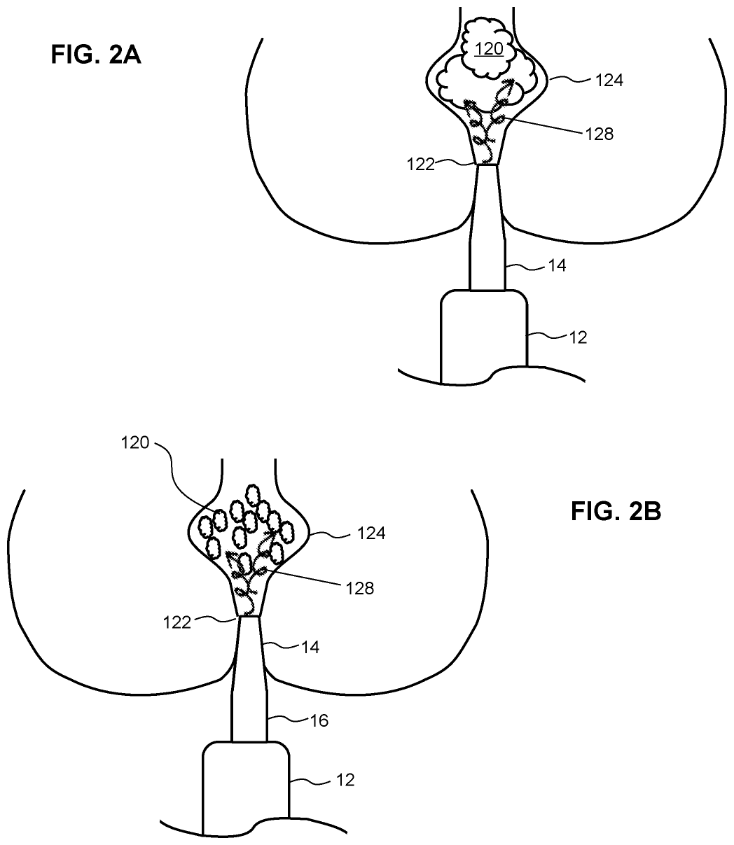

FIGS. 2A-2C show an example of how the rectal irrigation apparatus 10 could be operated. FIG. 2A shows the patient having a mass of hardened feces 120 (fecal impaction) in the rectum 124. The soft insertion tip 12 is inserted into the patient's anus 122. Irrigation fluid is pumped into the nozzle body 12 and sprayed out in a vortex fashion. The vortex irrigation spray 128 fragments the hardened feces 120.

FIG. 2B shows the results of the vortex irrigation spray 128. The feces 120 have been fragmented into smaller pieces. In FIG. 2C, the vacuum pump 52 is activated and the fragmented feces 120 are evacuated out of the rectum 124 and into the nozzle body 12. In the nozzle body 12, the helical auger 20 (not shown here) will further fragment the feces 120 so that it can be suctioned out through the discharge hose 32 and into the waste canister 40.

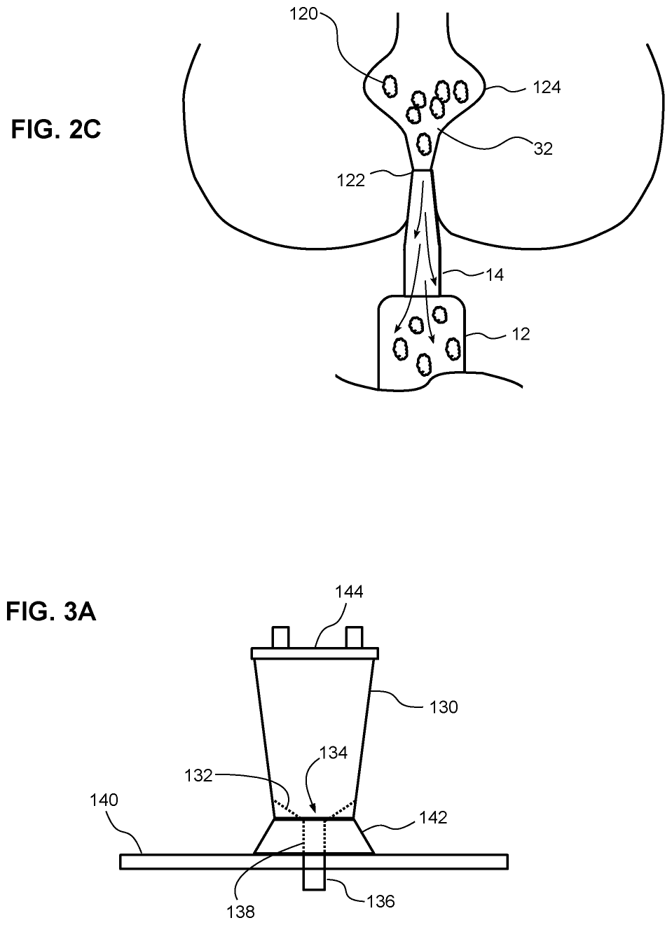

FIGS. 3A-3C show another design for a rectal irrigation apparatus of this invention with particular focus on the fecal waste section of the apparatus. FIG. 3A shows a detailed illustration of a design for a waste canister 130 used in this apparatus. Here, waste canister 130 has a lid 144 and a base 142. The waste canister 130 is set on top of a transparent hard plastic platform 140 (e.g. Plexiglass). The waste canister 130 has a sloped bottom 132 that allows the fecal waste to drain down into a discharge hole 134 at the floor of the waste canister 130. The fecal waste then passes through a drainage channel 138 in the base 142 of the waste canister 130. The fecal waste then exits out by gravity from a purge port 136 that extends downward from the bottom of the waste canister 130. FIG. 3B shows a perspective view of waste canister 130 and platform 140 in isolation. Platform 140 has a hole 134 through which purge port 136 is inserted therethrough.

FIG. 3C shows an example of how the fecal waste section of the apparatus could operate. Platform 140 is set on top of the toilet seat 154 of a flush toilet 150. Waste canister 130 is set on top of platform 140 with outlet port 136 inserted into hole 134 of platform 140. There is an electro-mechanical flush actuator 160 attached to flush toilet 150. Flush actuator 160 has an arm 162 that is set against the flush handle 152 of toilet 150. In operation, the fecal waste is collected into waste canister 130 and from there, drains out of purge port 136 and into the toilet 150. When the procedure is completed, the control module sends an actuation signal to flush actuator 160 via control line 164. This causes arm 162 to pivot down and press flush handle 152. Thus, the fecal waste is automatically flushed out of the toilet 150.

FIG. 4 shows a different example of a control module 100 of the invention in more detail. The user or an assistant may interact with this control module 100 to operate the rectal irrigation apparatus 10 described above. Control module 100 has buttons for operating various components of the apparatus 10. Button 92 activates or deactivates the water pump 50 for pumping the irrigation fluid. Pressing buttons 116 and 118 incrementally increase or decrease the water pump speed or flow rate of the irrigation fluid. Display window 110 shows the water pump speed or the flow rate.

Button 94 activates or deactivates the vortex motor 24. Corresponding (+) and (−) buttons incrementally increase or decrease the speed of the vortex motor 24. Display window 112 shows the vortex motor speed. Button 96 activates or deactivates the vacuum pump 52. Corresponding (+) and (−) buttons incrementally increase or decrease the amount of suction being applied. Display window 114 shows the amount of vacuum suction being applied.

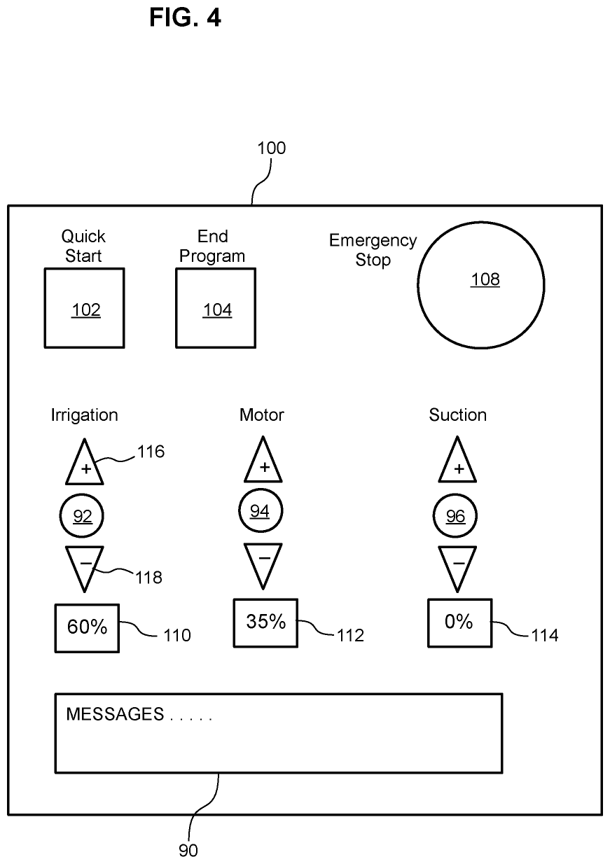

Control module 100 also has a quick start button 102. Pressing this button activates the water pump 50 to bring irrigation fluid into the nozzle body 12. After a short delay to allow filling of the nozzle body 12 with irrigation fluid, the vortex motor 24 is automatically activated. The water pump 50 is set to provide a flow rate of about 800 ml/min. The user/assistant may then adjust the flow of irrigation fluid or amount of vortex by pressing the relevant (+) or (−) buttons, as described above. In situations where a colon catheter is also being used (see FIG. 5 as explained below) and needs to be supplied with irrigation fluid, the water pump 50 is set to provide a total flow rate of about 1,050 ml/min for both the colon catheter and the spray nozzle.

Then after another short delay, as the patient's rectum 124 begins filling will irrigation fluid, the vacuum pump 52 is activated to suction out the waste irrigation fluid into the waste canister 40. The vacuum pump 52 is set to provide a suction pressure of 7.4 psi (51 kPa). This can be adjusted up or down as needed by pressing the relevant (+) or (−) buttons, as described above. Control module 100 may operate to balance the irrigation volume with the evacuation volume. For example, the inflow port 26 and the outflow port 30 on the spray nozzle may have flow sensors that are read by control module 100. Control module 100 could balance these inflow and outflow rates by adjusting the operation of the water pump 50, vacuum pump 52, or valves in the flow network.

Control module 100 also has a programmed stop button 104 to wind down the operation of the apparatus 10 for completing the irrigation procedure. Pressing button 104 deactivates the water pump 50 to stop the flow of irrigation fluid. Meanwhile, vortex motor 24 is allowed to continue operating to sustain the vortex action inside the nozzle body 12 and continue breaking up the fecal waste. Suction continues to be applied to empty out the nozzle body 12. The vortex motor 24 is then deactivated as nozzle body 12 empties. The vacuum pump 52 is deactivated when a fluid flow sensor detects no further flow out of the nozzle body 12 or a pressure sensor indicates no further resistance to the suction. There is also a big red button 108 for emergency stop of the apparatus 10, immediately deactivating the vortex motor 24, water pump 50, and vacuum pump 52. Relevant to the alternate apparatus design shown in FIG. 3C, the control module 100 could optionally also have a toilet flush button that activates the toilet flushing mechanism.

FIG. 5 shows a partial view of an alternate embodiment of the rectal irrigation apparatus that can provide a colonic enema via a colonic catheter 46 in addition to rectal irrigation. This partial view focuses on the configuration of the supply hose 34. The pumped irrigation fluid flows into the nozzle body 12 (not shown) via inflow port 26. In this alternate design, the apparatus also has a two-way valve 54 on the supply hose 34. The valve 54 can be set to allow flow of irrigation fluid into an accessory hose 56, which has an adapter 58. The colonic catheter 46 is connected to the adapter 58. The colonic catheter 48 has a distal opening 48 from which the irrigation fluid is streamed out. The colonic catheter 48 has a length of about 20 inches (51 cm) so that it can be positioned in the upper rectum or sigmoid colon.

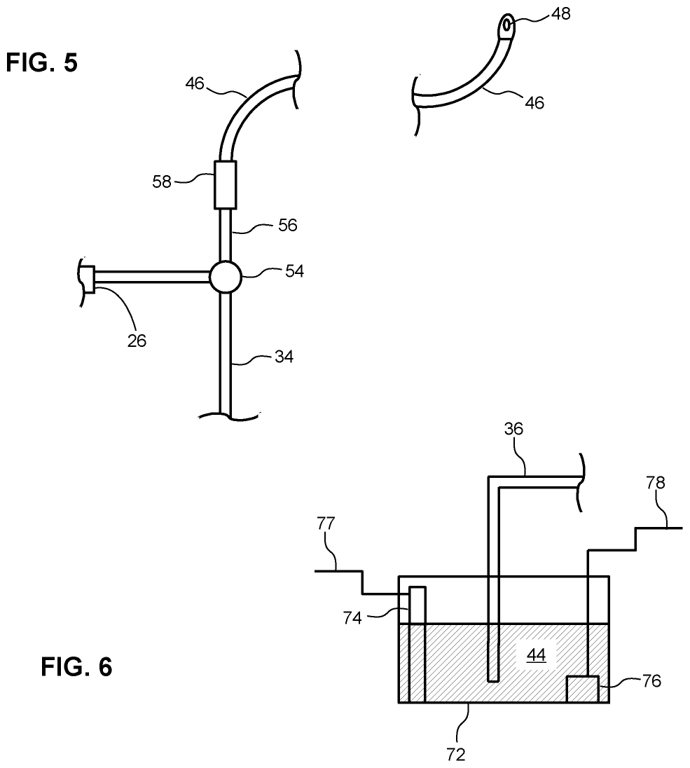

FIG. 6 shows a partial view of an alternate embodiment of the apparatus that has more useful features for a supply reservoir 72. In this design for supply reservoir 72, there is a fluid level sensor 74 to monitor the amount of irrigation fluid 44 remaining in the supply reservoir 72. The readings from sensor 74 are transmitted to the control module via sensor line 77. The supply reservoir 72 also has a heater unit 76 to warm the irrigation fluid 44 for improved comfort. The supply reservoir 72 also has a temperature sensor (not shown) to monitor the fluid temperature and transmit the readings to the control module. The control module adjusts the heater unit 76 to maintain a desired warm temperature (e.g. 103° F.) for the irrigation fluid 44.

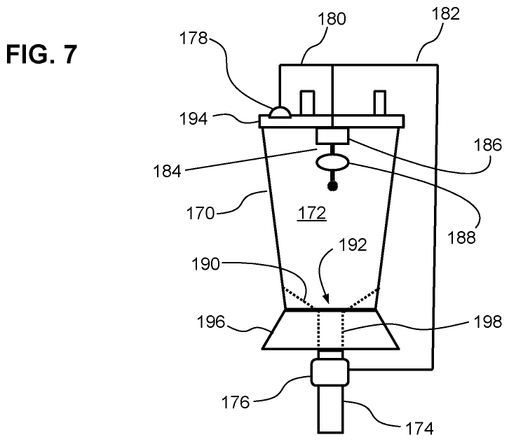

FIG. 7 shows another design for a waste container of this invention. Shown here is a waste canister 170 that could be used in the apparatus of this invention. Waste canister 170 has a lid 194 and a base 196. Waste canister 170 is designed to be set on top of a platform, such as the platform 140 shown in FIG. 3B. The waste canister 170 has a sloped bottom 190 that allows the fecal waste to drain down into a discharge hole 192 at the floor of the waste canister 170. The fecal waste then passes through a drainage channel 198 in the base 196 of the waste canister 170. The fecal waste then exits out by gravity from a purge port 174 that extends downward from the floor of the body 172 of the waste canister 170.

Waste canister 170 also has an air inlet valve 178 on the lid 194. During suction operation and standby situations, air inlet valve 178 is closed to seal waste canister 170 in air-tight condition. However, air inlet valve 178 is opened during purging of waste canister 170. This opening of air inlet valve 178 may be performed manually, but in this example embodiment, this is performed automatically. There is a float switch 184 that serves as a sensor for the fluid level inside waste canister 170. As the fluid level rises, the float bladder 188 on float switch 184 rises until it contacts the switch body 186. This causes float switch 184 to activate and open air inlet valve 178 (e.g. by electromechanical means). This also cause the float switch 184 to activate a purge valve 176 located on purge port 174. During suction operation and standby situations, purge valve 176 is closed. However, when activated by float switch 184, purge valve 176 opens to allow discharge of waste out through purge port 174 by gravity flow. Activation of float switch 184 may also turn off the vacuum suction operation.

Final Remarks: The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A rectal irrigation apparatus comprising:
a spray nozzle comprising a nozzle body and a soft nozzle tip;
an irrigation fluid container;
a water pump for pumping irrigation fluid out from the irrigation fluid container to the nozzle body, wherein the water pump has connections to the irrigation fluid container and the spray nozzle;
a vortex generator contained inside the nozzle body;
a waste container for receiving fecal waste, wherein the waste container has a connection to the spray nozzle;
a suction pump for suctioning fecal waste out of the nozzle body and discharging the fecal waste into the waste container, wherein the suction pump has a connection to the waste container;
a control module that is coupled to the water pump and the suction pump.

2. The apparatus of claim 1, further comprising an electric motor connected to the vortex generator, wherein the vortex generator is a spinning tool.

3. The apparatus of claim 1, further comprising a toilet flush actuator that is designed to actuate flushing of a toilet, wherein the toilet flush actuator is coupled to the control module.

4. The apparatus of claim 1, further comprising a toilet flush actuator that is designed to actuate flushing of a toilet, wherein the waste container comprises a fluid level or volume sensor, and wherein the toilet flush actuator is coupled to the fluid level or volume sensor.

5. The apparatus of claim 1, further comprising a colonic catheter tube, wherein the colonic catheter tube has a length of 38-102 cm and a width of 4-13.3 mm in outer diameter.

6. The apparatus of claim 1, wherein the control module includes a start program that causes:
activation of the water pump;
then after a delay, activation of the vortex generator;
then after a further delay, activation of the suction pump.

7. The apparatus of claim 1, wherein the soft nozzle tip has a diameter of 4-20 mm.

8. The apparatus of claim 1, further comprising a platform for the waste container, wherein the platform has a hole through which a purge port of the waste container passes therethrough, wherein the purge port is located at a bottom part of the waste container.

9. The apparatus of claim 1, wherein the control module has control interface elements for controlling the water pump and the suction pump.

10. The apparatus of claim 1, wherein the waste container comprises a purge port at a bottom location of the waste container.

11. A method of performing rectal irrigation on a patient, comprising:
having a rectal irrigation apparatus of claim 1;
having irrigation fluid in the irrigation fluid container;
inserting the soft nozzle tip of the spray nozzle into the rectum through the patient's anus;
operating the water pump to spray irrigation fluid out of the spray nozzle and into the rectum at a flow rate in the range of 400-1,500 ml/min;
activating the vortex generator, wherein the sprayed irrigation fluid has a swirling motion inside the rectum;
stopping or slowing the water pump to stop or slow the flow rate of irrigation fluid;
continuing to maintain the vortex generator for at least a partial duration while the water pump is stopped or slowed;
activating the suction pump to suction fecal waste out of the rectum, wherein the suction pump generates a suction pressure in the range of 5-100 kPa, and wherein the fecal waste is deposited into the waste container;
continuing to maintain the vortex generator for at least a partial duration while the suction pressure is applied.

12. The method of claim 11, wherein the apparatus further comprises an electric motor connected to the vortex generator, wherein activating the electric motor activates the vortex generator.

13. The method of claim 11, wherein the swirling motion of the irrigation fluid works to break-up or loosen the feces inside the rectum.

14. The method of claim 11, further comprising setting the waste container over a toilet.

15. The method of claim 14, wherein the waste container has a purge port located at a bottom of the waste container to allow outflow of fecal waste from the waste container, and the method further comprises discharging the fecal waste out of the purge port into the toilet by gravity flow.

16. The method of claim 15, further comprising sensing the fluid level or volume inside the waste canister.

17. The method of claim 16, wherein the purge port has a valve to control the flow through the purge port, and wherein the valve is opened when the fluid level or volume reaches a threshold level, thereby causing the fecal waste to drain out of the waste container into the toilet by gravity flow.

18. The method of claim 16, wherein the waste container has an air inlet port, and the air inlet port is opened when the fluid level or volume in the waste container reaches a threshold level.

19. The method of claim 11, wherein the apparatus further comprises a toilet flush actuator that is designed to actuate flushing of a toilet, wherein the toilet flush actuator is activated to flush the toilet when the irrigation procedure is completed.

20. The method of claim 15, wherein the apparatus further comprises a platform for the waste container, wherein the platform has a hole through which the purge port of the waste container passes therethrough, wherein the method further comprises:
setting the platform on a toilet;
setting the waste canister on the platform with the purge port passing through the hole.
